(19) United States
(12) Patent Application Publication
(10) Pub. No.: US 2013/0284176 A1
(43) Pub. Date: Oct. 31, 2013

(54) FORCE GAUGED CONTINUOUS POSITIVE AIRWAY PRESSURE NASAL INTERFACE

(71) Applicants: Benjamin D. Dickerson, Cincinnati, OH (US); Eric A. Liu, Westford, MA (US); Piotr C. Maniak, Chicago, IL (US); Janesh Lakhoo, Elk Grove Village, IL (US)

(72) Inventors: Benjamin D. Dickerson, Cincinnati, OH (US); Eric A. Liu, Westford, MA (US); Piotr C. Maniak, Chicago, IL (US); Janesh Lakhoo, Elk Grove Village, IL (US)

(21) Appl. No.: 13/843,669

(22) Filed: Mar. 15, 2013

Related U.S. Application Data
(60) Provisional application No. 61/619,699, filed on Apr. 3, 2012.

(51) Int. Cl. A61M 16/00

(52) U.S. Cl. CPC A61M 16/00
USPC 128/205.23; 128/204.18

(57) ABSTRACT

Disclosed are devices and methods for continuous positive airway pressure (CPAP) devices and related medical devices for treating patients susceptible to respiratory illnesses, including respiratory distress syndrome and sleep apnea. The device and methods employ force gauged CPAP nasal interface devices for these purposes.

### CROSS-REFERENCE TO RELATED APPLICATIONS

This application claims benefit of priority under 35 U.S.C. 119 to U.S. provisional patent application Ser. No. 61/619,699, filed Apr. 3, 2012, and entitled “FORCE GAUGED INFANT CPAP NASAL INTERFACE,” the contents of which are herein incorporated by reference in its entirety.

### BACKGROUND OF THE INVENTION

1. Field of the Invention

The present invention relates to continuous positive airway pressure (CPAP) devices and related medical devices for treating patients susceptible to respiratory illnesses, including respiratory distress syndrome and sleep apnea.

2. Discussion of the Art

Respiratory Distress Syndrome (RDS) occurs in infants whose lungs have not yet fully developed. The disease is mainly caused by a lack of a protective substance called surfactant, which helps the lungs inflate with air and keeps the air sacs from collapsing. Surfactant normally appears in fully developed lungs but not in infants with RDS. RDS can cause sleep apnea, a condition where an infant stops breathing during sleep for an intermittent period of time. Similar respiratory illnesses in the form acute respiratory distress syndrome and obstructive sleep apnea also affect adults.

A conventional method for treating RDS in infants and sleep apnea in both infants and adults is through use a Continuous Positive Airway Pressure (CPAP) device. CPAP devices deliver a constant, slightly pressurized, and sometimes humidified supply of air to the infant to ensure a continuous sustainable lung function.

Common CPAP devices are positioned to an infant's nasal passages in an inconsistent and often forceful manner. The force applied to the nasal passages frequently leads to pressure sores, broken facial bones, and stunted skeletal development in infants. Studies have shown that infants who received constant positive airway pressure using a CPAP device suffered moderate nasal injuries approximately thirty two percent of the time, and suffered severe nasal injuries twenty five percent of the time.

Constant positive airway pressure (CPAP) can be delivered through various types of devices and generators. Most CPAP devices include one or two air tubes that attach to an interface. The generator then attaches to a nasal adaptor. The nasal adaptor is sized according to the facial structure of the infant. In many CPAP devices, nasopharyngeal prongs that span from the nares to the nasopharynx are used. These devices interact directly with the infants nasal passages, and are often referred to as nCPAP devices. Although these nCPAP devices fulfill the purpose of providing the infant with constant positive airway pressure, often times these devices cause serious injury to the infant. The force with which an nCPAP device is positioned to the infants face varies considerably. In some cases a tremendous amount of variable force is produced, and because of an infant's extremely delicate facial tissue, this force commonly results in serious injury.

### BRIEF SUMMARY OF THE INVENTION

In a first aspect, a force gauged CPAP device is disclosed. The device includes a housing assembly, a spring, a retention member and a generator. The spring is adapted for coupling to an interior distal wall member of the housing assembly. The generator is disposed slideably within the housing assembly. The retention member maintains the generator within the housing assembly. The spring is adaptively compressed when the generator slideably engages the housing assembly.

In one respect of the first aspect, the device is used in a method for treating a patient suffering from a respiratory illness. According to the method, the device is administered to the patient.

In a second aspect, force gauged housing assembly for a CPAP generator is disclosed. The assembly includes a housing assembly, a spring and a retention member. The spring is adapted for coupling to an interior distal wall member of the housing assembly The housing assembly is adapted to permit a generator to be slideably disposed within the housing assembly. The retention member is adapted to maintain a generator within the housing assembly. The spring is adapted for compression when a generator slideably engages the housing assembly.

In one respect of the second aspect, the assembly is used in a method of using a force gauged CPAP device. The method includes four steps: providing a force gauged housing assembly according to the second aspect; providing a CPAP generator; assembling the CPAP generator into the force gauged housing assembly to provide force gauged CPAP device; and attaching the force gauged CPAP device to a user.

In another respect of the second aspect, the assembly is used in a method for treating a patient suffering from a respiratory illness. The method includes two steps: assembling a CPAP device from a force gauged housing assembly according to the second aspect and a generator; and administering the assembled force gauged CPAP device to the patient.

In a third aspect, a maximum force indicator for a CPAP device is disclosed. The indicator includes a plurality of supporting members; an assembly having a base member and two side wall members, wherein the two side wall members are non-displaceably coupled to the base member, and wherein a plurality of cavities are disposed in the distal portion of the assembly to receive the plurality of supporting members; a distal wall member having on its inner wall surface at least one push-block member and the plurality of supporting members attached thereto, wherein the distal wall member is displaceably coupled from the assembly; and a retention member, wherein the retention member maintains the assembly in proximity to a generate when present.

In one respect of the third aspect, the indicator is used in method with a force gauged CPAP device. The method includes four steps: providing a maximum force indicator of claim 21; providing a CPAP generator; assembling the CPAP generator into the maximum force indicator to provide force gauged CPAP device; and attaching the force gauged CPAP device to a user, wherein the operation of the maximum force gauge indicator informs the user about the maximum force gauge levels of CPAP output.

In another respect of the third aspect, the indicator is used in a method of treating a patient suffering from a respiratory illness. The method includes two steps: assembling a CPAP device from a maximum force indicator and a generator; and administering the assembled force gauged CPAP device to the patient.

In a fourth aspect, a device adapted to gauge an amount of force applied to a patient during a CPAP nasal insert insertion procedure is disclosed. The device includes a housing assembly, a generator and a spring. The housing assembly includes two vertical side wall members and a vertical distal wall member. The vertical wall member has a spring retention element. The generator includes a distal wall member having an outer surface and two vertical side wall members each having an outer surface, and said generator is translationally engaged to said housing assembly. The spring is disposed in said spring retention element so that said spring is in force communication with said housing assembly and said generator. The generator further comprises a visual indicator positioned for readability on at least one vertical side wall member outer surface, said visual indicator being calibrated to said spring and configured for gauging an amount of force applied to a patient during a CPAP nasal insert insertion procedure.

In a fifth aspect, a system for applying a CPAP nasal insert to a patient is disclosed. The system includes a CPAP nasal insert and a device. The device includes a housing assembly, a generator and a spring. The housing assembly includes two vertical side wall members and a vertical distal wall member, wherein said vertical wall member has a spring retention element. The generator includes a distal wall member having an outer surface and two vertical side wall members each having an outer surface. The generator is translationally engaged to said housing assembly. The spring is disposed in said spring retention element so that said spring is in force communication with said housing assembly and said generator. The generator further comprises a visual indicator positioned or readability on at least one vertical side wall member outer surface. The visual indicator is calibrated to said spring and configured for gauging an amount of force applied to a patient during a CPAP nasal insert insertion procedure.

In a sixth aspect, a method for applying a CPAP nasal insert to a patient during a CPAP nasal insert insertion procedure is disclosed. The method includes the following steps. The first step is providing a nasal insert. The second step is providing a device for gauging the amount of force applied to the patient during a CPAP nasal insert insertion procedure. The third step is contacting the nasal insert with the device for gauging the amount of force applied to a patient during a CPAP nasal insert insertion procedure. The fourth step is contacting the combined nasal insert and device for gauging the amount for force applied to a patient during a CPAP nasal insert insertion procedure with the patient. The fifth step is compressing the combined insert and device to an amount no greater than determined safe by the visual indicator that is placed on an outward facing surface of the device. The final step is securing straps interwoven with the device to hold the combined nasal insert and device in place on the patient.

### BRIEF DESCRIPTION OF THE DRAWINGS

FIG. 1 depicts a schematic illustrating an exploded perspective view of one preferred embodiment.

FIG. 2 depicts a schematic illustrating a perspective view of one preferred embodiment similar to that presented in FIG. 1 in assembled form.

FIG. 3 depicts a schematic illustrating an exploded perspective view of one preferred embodiment.

FIG. 4 depicts a schematic illustrating a perspective view of one preferred embodiment similar to that presented in FIG. 3 in assembled form.

FIG. 5 depicts a schematic illustrating a perspective view of one preferred embodiment having a no rail members.

FIG. 6A depicts a schematic illustrating an exploded perspective view of one preferred embodiment of a maximum force indicator (assembled with generator).

FIG. 6B depicts a schematic illustrating an exploded perspective view of one preferred embodiment of a maximum force indicator (generator fully compressed).

FIG. 6C depicts a schematic illustrating an exploded perspective view of one preferred embodiment of a maximum force indicator (generator relaxed).

FIG. 7A depicts a comparison of Force data for prototype and Airlife device, wherein the graph depicts a box plot of the peak force data.

FIG. 7B depicts a comparison of Force data for prototype and Airlife device, wherein the graph depicts histograms of the peak force data.

FIG. 7C depicts a comparison of Force data for prototype and Airlife device, wherein the depicts a box plot of the steady state force data.

FIG. 7D depicts a comparison of Force data for prototype and Airlife device, wherein the graph depicts histograms of the steady state force data.

### DETAILED DESCRIPTION OF THE INVENTION

In the following detailed description, reference is made to the accompanying figures, which form a part hereof In the figures, similar symbols typically identify similar components, unless context dictates otherwise. Insofar as possible, like parts and modules have the same reference numeral in the figures. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

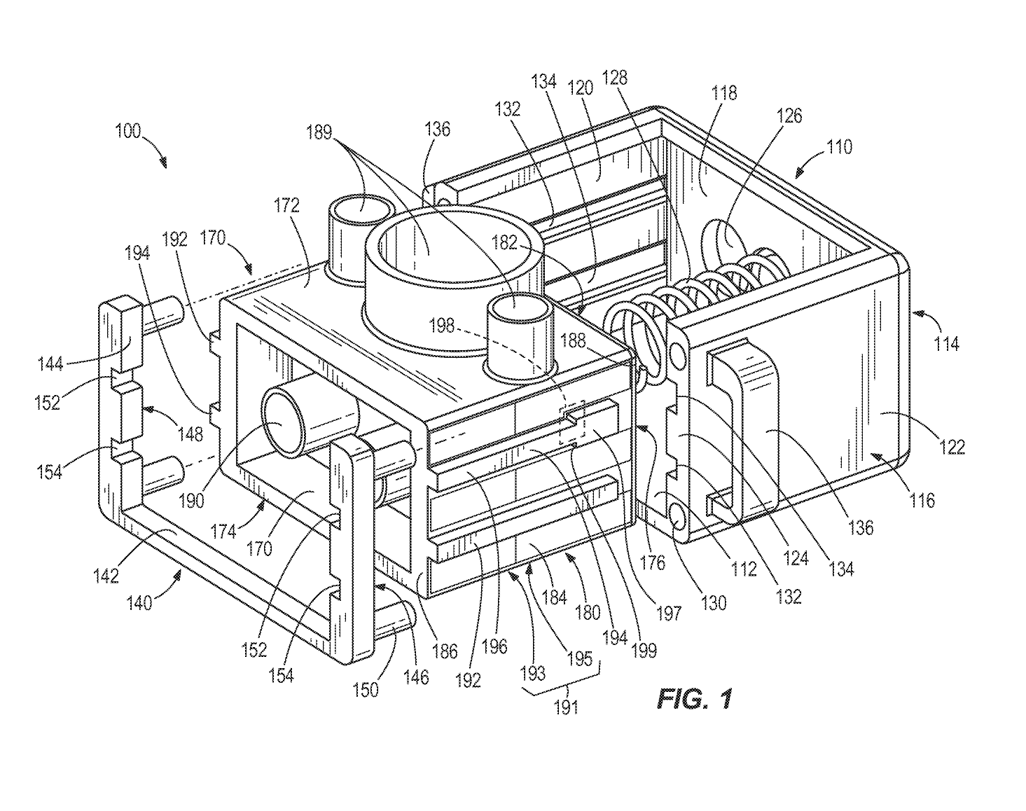

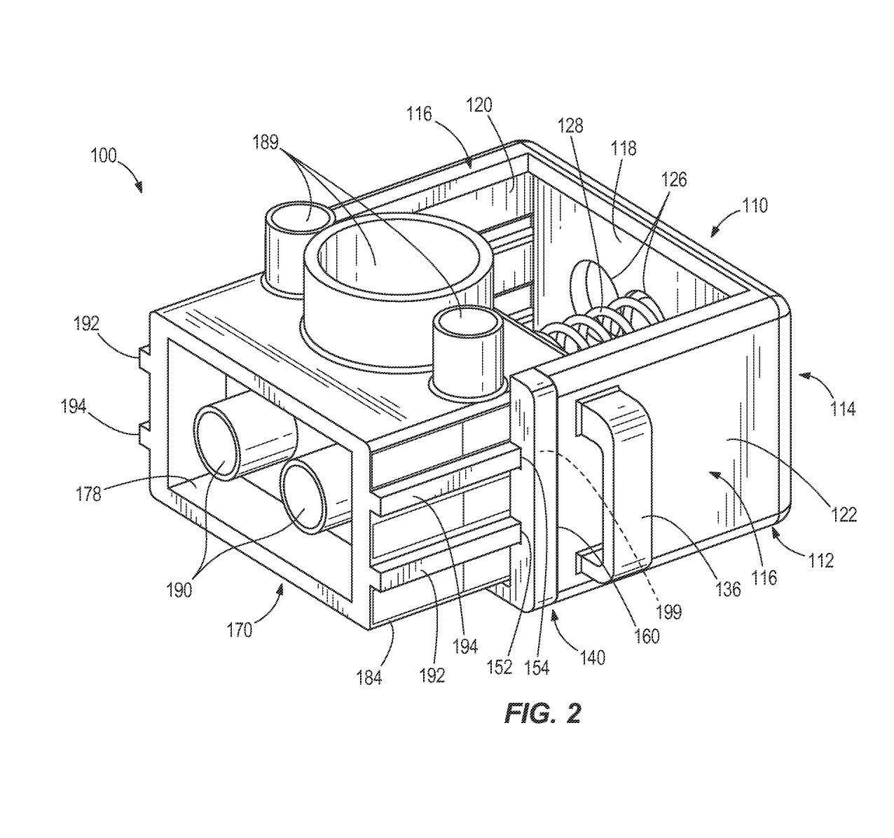

One preferred embodiment is illustrated in FIGS. 1 and 2. In FIG. 1, device 100 includes housing assembly 110, retention cap 140 and generator 170. In FIG. 2, device 100 is assembled with generator 170 located inside housing assembly 110 with retention cap 140 attached to housing assembly 110 at interface 160.

Housing assembly 110 of device 100 preferably includes the following features. Housing assembly 110 includes horizontal base member 112, vertical distal wall member 114 and two vertical side wall members 116. Horizontal base member 112 is preferably substantially plain surface. Vertical distal wall member 114 includes an inner wall surface 118. Vertical side wall members 116 include an inner wall surface 120, an outer wall surface 122 and a proximal surface 124.

Spring retention element 126 accommodates spring 128. Spring retention element 126 can be any structure amenable for retaining spring 128, such as a recess; a pocket; one or more rods or like structures; immobilized tie-downs, such as clips, staples, sutures, tapes, wires, rivets and the like; an adhesive, such as glue, epoxy and the like; among others. Spring retention element 126 is preferably a recess located within inner wall surface 118. Spring 128 is preferably in continuous mechanical communication with the housing assembly 110 during operation of device 100.

A plurality of pin holes 130 are preferably located on proximal surface 124 of vertical side wall members 116. Pin holes 130 can be of any shape and size, provided that pin holes 130 mate with pins 150 of retention cap 140 to ensure securely fastened interface 160. Preferably, pin holes 130 are substantially round in shape.

Housing assembly 110 preferably includes one guide groove 132 and one stop groove 134 located on inner surface 120 of each vertical side member 116. Both guide groove 132 and stop groove 134 run along the inner wall surface 120 and terminate at proximal surface 124 and preferably at the junction of inner wall surface 120 with inner wall surface 118.

Grooves 132 and 134 can be located at any position along the dimension of vertical side member 116 of housing assembly 110, provided that the position(s) of grooves 132 and 134 do not interfere with the structural integrity of pin holes 130. Thus, in the embodiment of device 100 illustrated in FIG. 1, grooves 132 and 134 can have the inverse relative orientation on each vertical side member 116. For example, where guide groove 132 is positioned below stop groove 134 on the vertical dimension of one vertical side member 116 (see FIG. 1, positions of grooves 132 and 134 on right vertical side member 116), guide groove 132 is positioned above stop groove 134 on the vertical dimension of the opposing vertical side member 144 (see FIG. 1, positions of grooves 132 and 134 left vertical side member 116).

In alternate embodiments of device 100, grooves 132 and 134 can have the same relative orientation on each vertical side member 116 of housing assembly 110. For example, guide groove 132 is positioned below stop groove 134 on the vertical dimension of both vertical side members 116.

Referring to FIG. 1, grooves 132 and 134 preferably have dissimilar sizes. Guide groove 132 preferably has a substantially uniform size and shape throughout its length that is smaller than the corresponding size and shape of stop groove 134. The reason for stop groove 134 having an increased size throughout its length relative to guide groove 132 is attributed to the fact that stop groove 134 accommodates a larger rail than that which guide groove 132 accommodates. Yet the shapes and sizes of grooves 132 and 134 need not be identical for their like counterparts on the opposing vertical side member 116.

Referring to FIG. 2, preferred features of grooves 132 and 134 of housing assembly 110 are that they form preferably substantially aligned, continuous, extended grooves with grooves 152 and 154 of retention cap 140 when housing assembly 110 when is mated with retention cap 140 at interface 160. In particular, guide grooves 132 of housing assembly 110 and guide grooves 152 of retention cap 140 together preferably form an extended guide groove when housing assembly 110 and retention cap 140 are mated at interface 160. Likewise, stop grooves 134 of housing assembly 110 and stop grooves 154 of retention cap 140 together form an extended stop groove when housing assembly 110 and retention cap 140 are mated at interface 160. A substantially aligned, continuous and extended glide groove and extended stop grove is formed in device 110 if generator 170 configured with corresponding guide rails and stop rails can translationally slide with ease (that is, without substantial frictional contact or force being applied) along at least a portion the extended guide groove and extended stop groove at interface 160, at least until the until stop rail blocker 199 encounters stop groove 154.

Referring to FIG. 1, housing assembly 110 also includes head-strap element 136 positioned on outer wall surface 122 of vertical wall members 116.

Retention cap 140 of device 100 preferably includes the following features. Retention cap 140 includes a horizontal base member 142 and two vertical side members 144. Vertical side members 144 include distal surface 146 that contacts housing assembly 120 within assembled device 100 and inner side surface 148 that contacts generator 160 within assembled device 100.

Retention cap 140 preferably includes a plurality of pins 150 located on distal surface 146 of each vertical side member 144. Pins 150 can be of any shape and size, provided that pins 150 mate with pin holes 130 of housing assembly 110 to ensure securely fastened interface 160. Preferably, pins 150 are substantially round in shape.

Retention cap 140 preferably includes one guide groove 152 and one stop groove 154 located on inner surface 150 of each vertical side member 144 of retention cap 140. Grooves 152 and 154 extend preferably throughout the width of inner surface 150. Grooves 152 and 154 can be located at any position along the dimension of vertical side member 144 of retention cap 140, provided that the position(s) of grooves 152 and 154 do not interfere with the structural integrity of pins 150. Thus, in the embodiment of device 100 illustrated in FIG. 1, grooves 152 and 154 can have the inverse relative orientation (termed “offset”) on each vertical side member 144. For example, where guide groove 152 is positioned below stop groove 154 on the vertical dimension of one vertical side member 144 (see FIG. 1, positions of grooves 152 and 154 on right vertical side member 144), guide groove 152 is positioned above stop groove 154 on the vertical dimension of the opposing vertical side member 144 (see FIG. 1, positions of grooves 152 and 154 left vertical side member 144).

In alternate embodiments of device 100, grooves 152 and 154 can have the same relative orientation on each vertical side member 144 of retention cap 140. For example, guide groove 152 is positioned below stop groove 154 on the vertical dimension of both vertical side members 144.

Referring to FIG. 1, grooves 152 and 154 can have substantially the same shape and size, which represents a preferred embodiment. Yet the shapes and sizes of grooves 152 and 154 need not be identical to one another or to their like counterpart on the opposing vertical side member 144. For example, guide grooves 152 can differ with respect to each other and to stop groove 154 in terms of their respective shape (for example, square, rectangular, triangular, ovoid, etc.) and size (longer, deeper). Likewise, stop grooves 154 can differ with respect to each other and to guide groove 152 in terms of their shape and size.

Preferred features of grooves 152 and 154 of retention cap 140 are that they form preferably substantially aligned, continuous and extended grooves with grooves 132 and 134 of housing assembly 110 when retention cap 140 is mated with housing assembly 110 at interface 160. Thus, guide grooves 152 of retention cap 140 and guide grooves 132 of housing assembly 110 together preferably form an extended guide grooves when the retention cap 140 and housing assembly 110 are mated at interface 160. Likewise, stop grooves 152 of retention cap 140 and stop grooves 134 of housing assembly 110 together preferably form an extended guide groove when retention cap 140 and housing assembly 110 are mated at interface 160. A substantially aligned, continuous and extended glide groove and extended stop grove is formed in device 110 if generator 170 configured with corresponding guide rails and stop rails can translationally slide with ease (that is, without substantial frictional contact or force being applied) along at least a portion the extended guide groove and extended stop groove at interface 160, at least until stop rail blocker 199 encounters stop groove 154.

Generator 170 of device 100 preferably includes the following features. Generator 170 includes a top surface member 172, a bottom surface 174, a distal wall member 176, a proximal wall recess member 178 and two vertical side wall members 180. Distal wall member 176 includes outer surface 182. Vertical side wall members 180 include outer surface 184, proximal surface 186 and distal surface 188.

Operational structures of generator 170 pertaining to CPAP functions are well known in the art and only briefly described herein. Air channel adaptors 189 are located and extend through top surface member 172. Air channel adaptors 189 provide, in part, fluid communication with common gas supply tubes via tubing (not shown) to supply an appropriately humidified O₂/N₂ gas mixture to the interior of generator 170. Nasal insert outlets 190 are located and extend through the proximal wall recess member 178. Nasal insert outlets 190 provide an appropriately humidified O₂/N₂ gas mixture from the generator 170 to the patient.

Referring to FIG. 1, generator 170 preferably includes the following additional features as they relate to housing assembly 110 and retention cap 140. Outer surface 182 of distal wall member 176 is preferably in continuous mechanical communication with spring 128 during operation of device 100.

Generator 170 preferably includes one guide rail 192 and stop rail 194 located on outer surface 184 of each vertical side member 180. Rails 192 and 194 extend preferably along substantially the entire length of outer surface 184, thereby extending from proximal surface 186 and to near distal surface 188. Rails 192 and 194 can be located at any position along the dimension of vertical side member 180 of generator 170.

Referring to FIG. 1, rails 192 and 194 preferably have a dissimilar shape and size along at least a portion of their respective lengths. Guide rail 192 can have a substantially uniform size and shape throughout its length. Stop rail 194 includes a first stop sub-rail 196, a second stop sub-rail 197 and an interface stop rail region 198 that is located between stop sub-rails 196 and 197. Stop sub-rail 196 terminates at proximal surface 186 and at interface rail region 198. Stop sub-rail 197 terminates at interface stop rail region 198 and preferably near distal surface 188. Stop sub-rail 196 and stop sub-rail 197 preferably have substantially uniform size and shape throughout their respective lengths; however, with respective sizes and shapes of stop sub-rails 196 and 197 are differ with respect to each other. Stop sub-rail 196 can have a size and shape similar to that of guide rail 192; however, stop sub-rail 197 is preferably larger than stop sub-rail 196. Interface stop rail region 198 can have an intermediate size and shape between those corresponding to stop sub-rails 196 and 198.

Guide rails 192 of generator 170 are configured by their size and shape to translationally slide freely (for example, without undue friction) in guide grooves 132 and 152 of housing assembly 110 and retention cap 140, respectively, in device 100. Stop sub-rails 196 is configured by their size and shape to translationally slide freely in stop grooves 134 and 154 of housing assembly 110 and retention cap 140, respectively, in device 100. However, stop sub-rails 197 are configured by their size and shape to translationally slide freely in only stop grooves 134 of housing assembly 110 and not stop groove 154 of retention cap 140 in device 100. Depending upon the size and shape of interface stop rail region 198, it may or may not translationally slide freely through stop groove 154 of retention cap 140. Blocker 199 defines the position on generator 170 along which stop rail 194 cannot translationally slide freely past stop groove 154 of retention cap 140 in device 100.

Having described the structure and function of the guide grooves and rails, as well as stop grooves and rails, of device 100 (FIG. 1), obvious permutations of their arrangement on housing assembly 110, cap assembly 140 and generator 170 will be readily apparent based upon this disclosure. Such permutations include switching which components of device 100 include rails vs. grooves. It will be apparent that rails rather than grooves can be incorporated onto both housing assembly 110 and retention cap 140, while grooves rather than rails can be incorporated onto generator 170.

As explained above, and with reference to FIG. 1, spring 128 is preferably in continuous mechanical communication with both housing 110 and generator 170 in device 100. In that particular embodiment, spring 128 has only attachment point, where one end of spring 128 that is preferably connected to inner wall surface 118 of housing assembly 110. In alternative embodiments, end of spring 128 can be connected to outer surface 182 of distal wall member 176 of generator 170 as its only attachment point with device 110.

In alternate embodiments of device 100, however, spring 128 can be in continuously mechanical communication with only one member selected from housing assembly 110 and generator 170. For example, embodiments of device 110 can include spring 128 suspended in housing assembly 110 by attachment of a midpoint of spring 128 to horizontal base member 112. Other embodiments of device 110 can include spring 128 attached to outer surface 182 of distal wall member 176 of generator 170.

In the foregoing embodiments, such connections and attachments can be accomplished using any structure amenable for connecting spring 128 to these and other features of housing assembly 110 and/or generator 170 (such as inner wall surface 118, horizontal base member 112 and outer surface 182, among others) such as a recess; a pocket; one or more rods or like structures; immobilized tie-downs, such as clips, staples, sutures, tapes, wires, rivets and the like; an adhesive, such as glue, epoxy and the like; among others. Spring retention element 126 is preferably a recess located within inner wall surface 118.

In yet another embodiment, device 100 can be manufactured with spring 128 integrated into either housing assembly 110 or generator 170. In such an embodiment, spring 128 can be a traditional spring that is insert molded into the plastics.

In other embodiment, spring 128 can be a living spring that is made of plastic as part of one of housing assembly 110 or generator 170. Spring 128 in this embodiment produces its force output as housing assembly 110 and generator 170 are slid together, and the interference forces the plastic to deflect.

Spring 128 can be a spring of any configuration and composition, including an air spring, a coil spring, a helical spring, a leaf spring, wave spring and a torsional spring.

However configured, for appropriate function within device 100 during active operation, spring 128 contacts both outer surface 182 of distal wall member 176 of generator 170 and inner wall surface 118 of housing assembly 110 to create compression between generator 170 and housing assembly 110 as a result of the force device 100 applies to the patient. Thus, spring 128 is in force communication with both housing assembly 110 and generator 170 during operation of device 100.

Referring to FIG. 2, device 100 is illustrated in assembled form. Pins 150 of retention cap 140 mate with pin holes 130 of housing assembly 110 to create securely fastened interface 160. Preferably, pins 150 are slightly larger than pin holes so that they mate together by press fit to create interface 160. An alternative approach can be used to create interface 160, such as the use of an adhesive material to fill pin holes 130 before insertion of pins 150.

As explained previously, and in reference to FIG. 1, the preferred arrangement of the guide grooves and stop grooves, as well as their mated guide rails and stop rails, is in inverse relative orientation relative to the opposite vertical side wall members 116 and opposite vertical side wall members 144. The offset arrangement of stop rails and stop grooves maintain the balance of generator 170 within housing assembly 110, and further limits binding and preventing incorrect assembly of device 100.

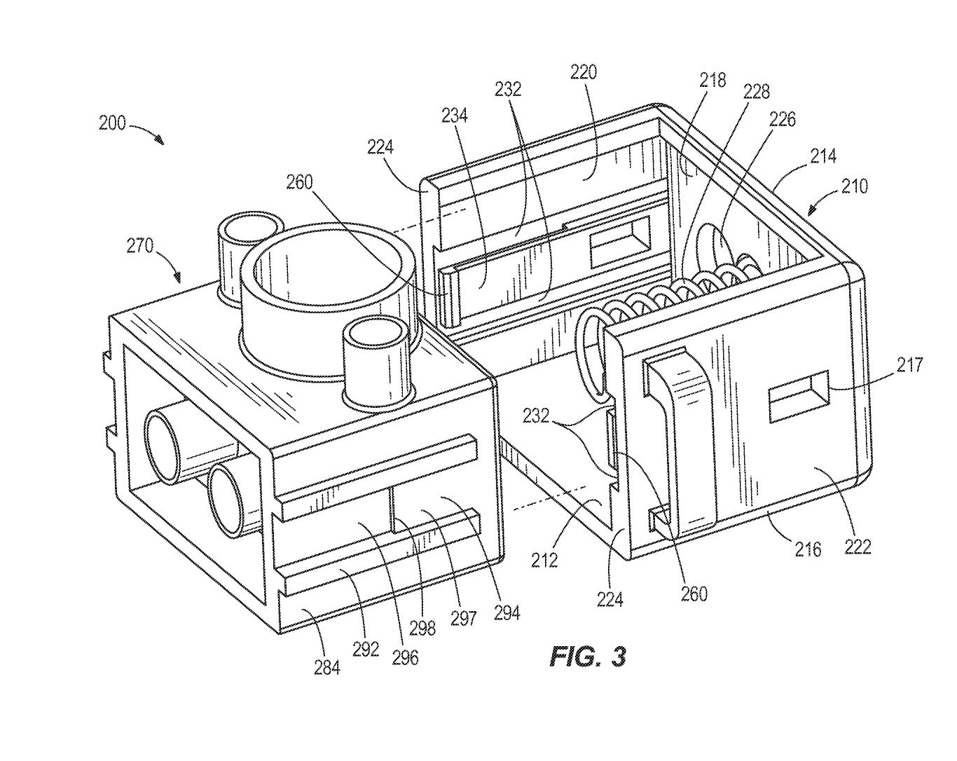

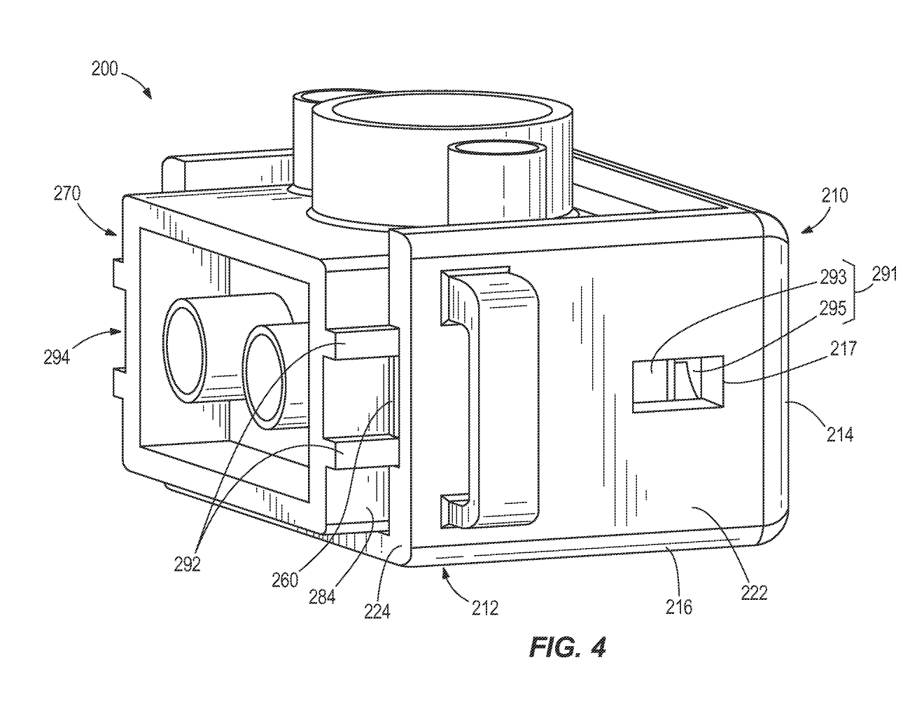

Another preferred embodiment is illustrated in FIGS. 3 and 4. In FIG. 3, device 200 includes housing assembly 210 and generator 270. In FIG. 4, device 200 is assembled with generator 270 located inside housing assembly 210, wherein generator 270 is interlocked to housing assembly 210 at retaining member 260.

Device 200 includes similar features as presented for device 100 of FIGS. 1 and 2, except that retention cap 140 of device 100 is not required for device 200.

Housing assembly 210 of device 200 preferably includes the following features. Housing assembly 210 includes horizontal base member 212, vertical distal wall member 214 and two vertical side wall members 216. Horizontal base member 212 is preferably substantially plain surface. Vertical distal wall member 214 includes an inner wall surface 218. Vertical side wall members 216 include inner wall surface 220, outer wall surface 222 and proximal surface 224.

Spring retention element 226 accommodates spring 228. Spring retention element 226 can be any structure amenable for retaining spring 228, such as a recess; a pocket; one or more rods or like structures; immobilized tie-downs, such as clips, staples, sutures, tapes, wires, rivets and the like; an adhesive, such as glue, epoxy and the like; among others. Spring retention element 226 is preferably a recess located within inner wall surface 218. Spring 228 is preferably in continuous mechanical communication with housing assembly 210 during operation of device 200.

Housing assembly 210 preferably includes two guide grooves 232 and one stop rail 234 located on inner surface 220 of each vertical side member 216. Both guide groove 232 and stop rail 234 run along the inner wall surface 220 and terminate at proximal surface 224 and preferably at the junction of inner wall surface 220 with inner wall surface 218. Retaining member 260 is positioned at the end of stop rail 234 terminated at proximal surface 224.

Grooves 232 can be located at any position along the dimension of vertical side member 216 of housing assembly 210. Grooves 232 preferably flank rail 234 on the vertical dimension (that is, one glide groove 232 lies above and below stop rail 234).

Referring to FIGS. 3 and 4, grooves 232 and rail 234 preferably have dissimilar sizes. Guide groove 232 preferably has a substantially uniform size and shape throughout its length that is smaller than the corresponding size and shape of stop rail 234. The reason for stop rail 234 having an increased size throughout its length relative to guide groove 232 is attributed to the fact that stop rail 234 includes retention member 260 that provides the interlock feature for positively holding generator 270 inside housing assembly 210. No such function is required of groove 232 in the embodiment of device 200.

Yet the shapes and sizes of grooves 232 and rail 234 need not be identical for their like counterparts on the opposing vertical side member 216. But the shapes, sizes and locations of guide groove 232 and stop rail 234 for a given vertical side member 216 are preferably compatible to match with the shapes, sizes and locations of guide rails 292 and stop groove 294 of generator 270. Such compatibility ensures that generator 270 can translationally slide freely relative to housing assembly 210 in device 200.

Generator 270 preferably includes two guide rail 292 and one stop groove 294 located on outer surface 284 of each vertical side member 280. Rails 292 and 294 extend preferably along substantially the entire length of outer surface 284, thereby extending from proximal surface 286 and to near distal surface 288. Rails 292 and groove 294 can be located at any position along the dimension of vertical side member 280 of generator 270. Rails 292 preferably flank groove 294 on the vertical dimension (that is, one glide rail 292 lies above and below stop groove 294).

Referring to FIG. 3, stop groove 294 preferably have a dissimilar shape and size along at least a portion of its length. Stop groove 294 includes a first stop sub-groove 296, a second stop sub-groove 297 and an interface stop groove region 298 that is located between stop sub-grooves 296 and 297. Stop sub-groove 296 terminates at proximal surface 286 and at interface rail region 298. Stop sub-groove 297 terminates at interface stop groove region 298 and preferably near distal surface 288. Stop sub-groove 296 preferably have substantially uniform size and shape throughout its length. Stop sub-groove 297 preferably has a progressively increasing thickness along its length, wherein the thickness of sub-groove 297 at the near distal surface 288 is negligible (being substantially contiguous with the surface of outer surface 284), and wherein thickness of sub-groove 297 at the interface stop groove region 298 is substantial. The thickness of sub-groove 297 at the interface stop groove region 298 is sufficiently substantial so as to interlock generator 270 into housing assembly 210 when retaining member 260 of housing assembly 210 passes across the interface stop groove region 298.

Guide rails 292 of generator 270 are configured by their size and shape to translationally slide freely (for example, without undue friction) in guide grooves 232 of housing assembly 210 in device 200. Stop sub-groove 296 is configured by their size and shape to translationally slide freely in stop rail 234 of housing assembly 210 in device 200. However, stop sub-groove 297 is configured by its size and shape to translationally slide freely in stop rail 234 only until to interface stop groove region 298 encounters retaining member 260 of housing assembly 110. Thus, interface stop groove region 298 acts as the functional equivalent of as blocker element because feature 298 defines the position on generator 270 beyond which generator 270 cannot translationally slide freely out of housing assembly 210 in device 200.

Once interlock between housing assembly 210 and generator 270 has occurred, device 200 is stably assembled for use. Device 210 can be readily disassembled by disengaging retention member 260 from interface stop groove region 298. For this purpose, retention member 260 of stop rail 234 has some flexibility to enable it to pass over interface stop groove region 298 for disengagement. Such disassembly is conveniently performed to provide servicing and cleaning functions to the underlying components. In general, however, device 200 is not disassembled during operation.

Having described the structure and function of the guide grooves and rails, as well as stop grooves and rails, of device 200 (FIGS. 3-4), obvious permutations of their arrangement on housing assembly 210 and generator 270 will be readily apparent based upon this disclosure. Such permutations include switching which components of device 200 include rails vs. grooves. It will be apparent that the appropriate combinations of rails and grooves can be incorporated onto both housing assembly 210 and generator 270 so that retaining member 260 is resides on generator 270 and that interface stop groove region 298 is incorporated on housing assembly 210.

### Rails: Alternative Embodiments.

Preferred embodiments feature rail elements on both sides of device subcomponents that are offset. The offset rails provide a benefit of preferred manufacturing process by preventing the device from being assembled incorrectly. In other embodiments, symmetrical rails that are not offset can be incorporated into device subcomponents.

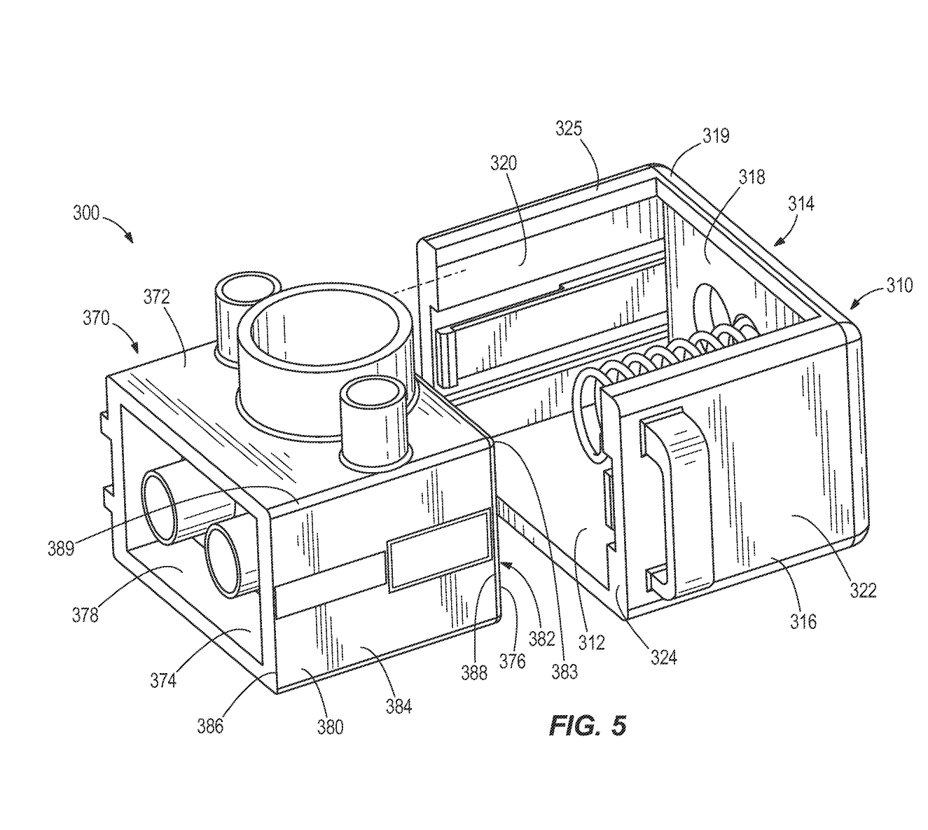

Referring to FIG. 5, embodiment of device 300 can be devoid of rails altogether. Housing assembly 310 of device 300 preferably includes the following features. Housing assembly 310 includes horizontal base member 312, vertical distal wall member 314 and two vertical side wall members 316. Horizontal base member 312 is preferably substantially plain surface. Vertical distal wall member 314 includes an inner wall surface 318 and top surface 319. Vertical side wall members 316 include inner wall surface 320, outer wall surface 322, a proximal surface 324, and top cap 325.

Generator 370 of device 300 preferably includes the following features. Generator 370 includes top surface member 372, bottom surface 374, distal wall member 376, a proximal wall recess member 378 and two vertical side wall members 380. Distal wall member 376 includes outer surface 382 and top surface 383. Vertical side wall members 380 include outer surface 384, proximal surface 386, distal surface 388 and top surface 389.

In this latter design implementation, top cap 325 would be required to contain generator 370 within housing assembly 310. A preferred design for this implementation includes top cap 325 on both vertical side wall members 316 of housing assembly 310 which preferably is in mechanical communication with top surface member 372 of generator 370 to retain generator 370 within housing assembly 310.

### Operational Indicator

Referring again to FIG. 1, outer surface 182 preferably includes indicator 191 having first indicator zone 193 and second indicator zone 195 arranged adjacent to one another. First indicator zone 193 is located in the proximal region of outer surface 182 while second indicator zone 195 is located in the distal region of outer surface 182. First indicator zone 193 preferably differs from second indicator zone 195 so that the patient or another attending the patient can determine readily and quickly whether device 100 is safely operating.

For example, common head-straps attached to head-strap elements 136 after device is attached to the patient's nasal passage are tightened around the patient's head. Indicator 191 is preferably used to determine the extent to which the head straps are tightened is based upon the relative force applied by the user compared to the tension created by spring 128. Thus, indicator 191 provides an indication to the user when the amount of force applied through the tightening of the head-strap is unsafe and likely to cause harm to the patient. As generator 170 is compressed into housing assembly 110, indicator 191 will slide from first indicator zone 193 to second indicator zone 195. First indicator zone 193 represents unsafe tension being applied to the head-strap while second indicator zone 195 represents safe tension being applied to the head-strap.

In yet another embodiment of device 100, indicator 191 can be placed on housing assembly 110 rather than on generator 170. Because generator of device 100 moves internally to housing assembly 110, translucent or optically transparent materials are preferably used for manufacturing housing assembly 110 so that movement of generator 170 relative to indicator 191 (and indicator zones 193 and 195) may be viewed through housing assembly 110.

Referring to FIGS. 3 and 4, embodiment of device 200 preferably includes viewing window 217 in at least one vertical side wall member 216 of housing assembly 210 to enable viewing of indicator 291 on generator 270. In this design, viewing window 217 on housing assembly 210 allows visualization of only one section of indicator 291 at once. This simplifies indicator 291 for the user, such that any appearance of second indicator zone 295 (for example, a color such as green or symbol for a “A-OKAY” marker) is an immediate warning of high forces, as opposed to lack of indicator zone 293 (for example, a color such as read or symbol for a “failing” marker). This also moves indicator 291 to the back of device 210 which prevents it from being obstructed or hidden by the head strap assembly attached at 236.

Indicator 191 (291) serves to inform attendants when high forces are being exerted on the patient's nasal septum as a result of the patient moving or device 100 (200) slipping while attached. Thus indicator 191 (291) provides a visual alarm critical to preventing prolonged exposure and potential breakdown of a patient's nasal septum.

Beside visual indicators, other embodiments of indicators include other sensory output implementations. For example, an audible indicator can provide one or more audible sounds that inform about device functions. A preferred audible indicator emits a sound that warns about unsafe operation conditions for the device. Another example of a sensory indicator is a vibration mode indicator. This type of indicator emits one or more vibrations preferably sensed by the patient. A preferred vibration mode indicator emits a vibration that warns about unsafe operation conditions for the device. Sensory indicators are preferably triggered by a motion sensor located on the device detecting the relative position of the generator relative to the housing assembly. Motion sensors having visual, audible, and vibrational outputs are well known in the art and can be incorporated into the devices disclosed herein.

### Force Gauged Assembly Enclosures for Commercial CPAP Generators

Embodiments disclosed herein are adaptable for use with generators produced from commercial manufactures. Accordingly, force gauged assembly enclosures having a plurality of features are within the scope of the devices disclosed herein. Those devices include a plurality of supporting members, such as guide elements, stop elements, spring elements, and visual indicator elements. Depending upon the generator manufacturer's requirements and generator implementations, force gauged assembly enclosures comprising any of the disclosed embodiments may be configured to provide the requisite force gauge measurement capabilities.

### Maximum Force Indicator

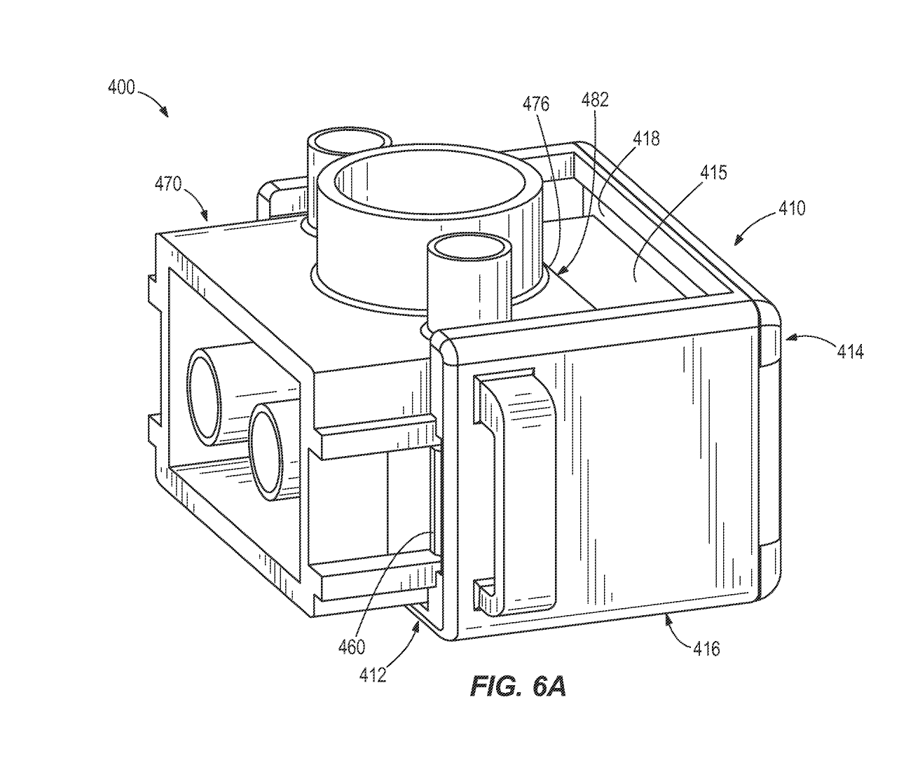

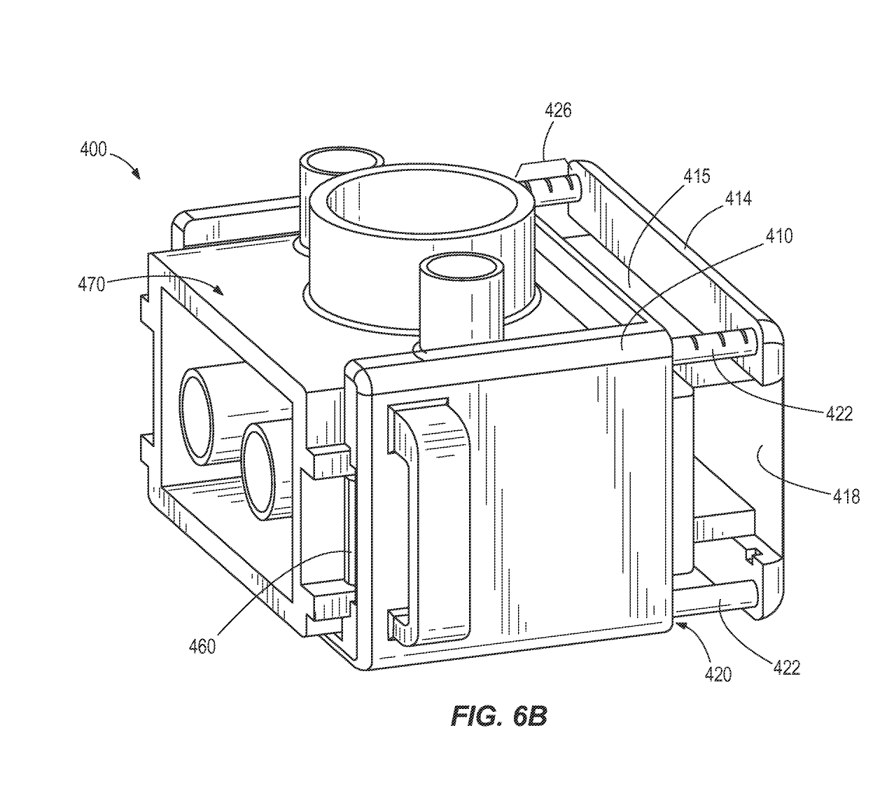

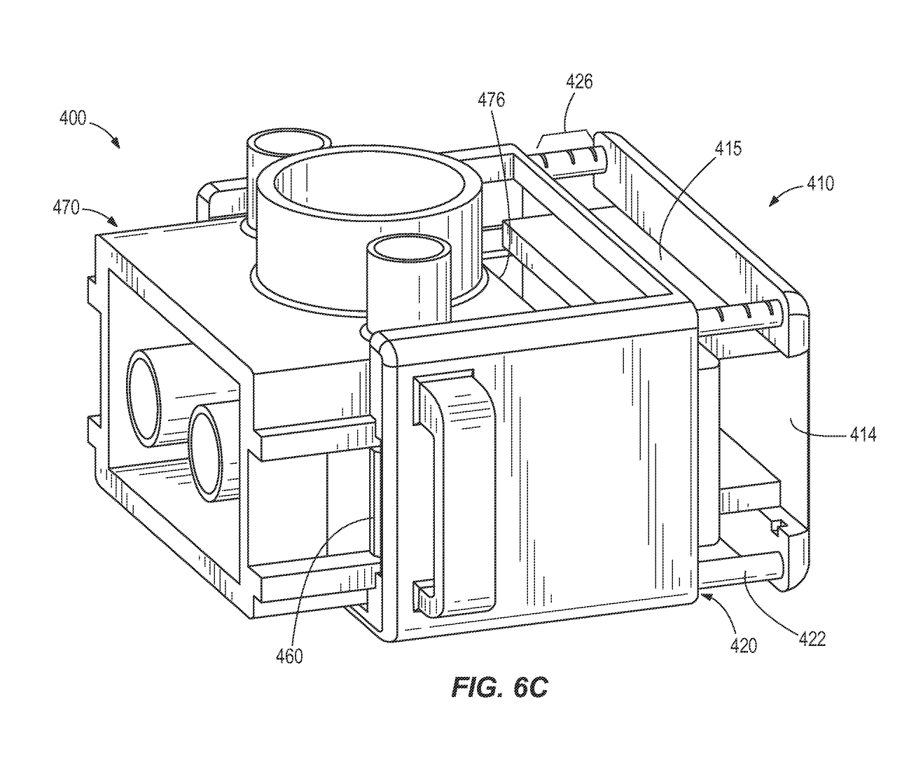

Referring to FIG. 6A, device 400 provides another embodiment of a force gauge device. Device 400 includes housing assembly 410 and generator 470.

Housing assembly 410 of device 400 preferably includes the following features. Housing assembly 410 includes horizontal base member 412, vertical distal wall member 414 and two vertical side wall members 416. Horizontal base member 412 is preferably substantially plain surface. Vertical distal wall member 414 includes an inner wall surface 418. At least one push-block member 415 is preferably coupled to inner wall surface 418. Push-block member 415 preferably established communication throughout the side-to-side length of distal vertical distal wall member 414. Push-block member 415 preferably is coupled to the outer surface 482 of distal wall member 476 of generator 470.

Referring to FIG. 6B, housing assembly 410 components vertical side wall members 416 and/or basement members 412 include at least two and preferably four more cavities 420 that preferably receive support rods 422. Distal vertical wall member 414 preferably displaceable from the remaining components of housing assembly 410, including horizontal base member 412 and both vertical distal wall members 414. Support rods 422 preferably are affixed to inner wall surface 418 and/or to base member 412. To couple distal vertical wall member 414 to horizontal base member 412 and both vertical distal wall members 414, support rods 422 are disposed into cavities 420. The support rods further include a plurality of measuring indications 426.

Housing assembly 410 thus includes a displaceable distal vertical wall member 416 that retains coupling to the remainder of housing assembly 410 by support rods 422 being disposed within cavities 420. The initial position of the distal wall member 416 is preferably proximal to base member 412 and side wall member 416.

Referring to FIG. 6A, assembly of generator 470 into housing assembly 410 preferably does not alter the location of distal wall member 416 relative to the base member 412 and side wall member 416. Likewise, low level compressions preferably do not displace distal wall member 414 from the remainder of housing assembly 410.

Referring to FIG. 6B, when generator 470 is compressed significantly into housing assembly 410, generator 470 enters the interior cavity of housing assembly 410, and distal wall member 476 of generator 470 actively pushes against push-block member(s) 415. Because push-block members 415 are coupled to distal wall member 414, the compression force of generator 470 preferably displaces distal wall member 414 away from the remainder of the housing assembly 410 by movement of the support rods 422.

The extent to which generator 470 exerts force against push-block(s) 415 will be directly related to the extent to which support rods 422 are exposed from cavities 420. The support rods 422 preferably have slight friction coefficient when disposed into cavities 420, so as to provide a memory effect of the extent to which the plurality of measuring indications 426 are exposed for counting. Thus, the support rods 422 remain extended even after generator 470 relaxes and the compression ceases (FIG. 6C).

Housing assembly 410 includes a retention member 460 to maintain generator 470 inside housing assembly 410. One preferred retention member 460 includes snap-fit interlocks with generator 470 similar to other interlocking features disclosed herein. For example, retaining member 460 located on the inner surface 420 of housing assembly 410 preferably can form the interlock by passing across a suitable opposing snap-fit feature located on the outer surface interface stop groove region 298 located on an outer surface 284 of vertical wall 280 of generator 470. Another preferred retention member 460 includes a retention cap, like cap 140.

### Manufacturing Considerations: Best Mode

Embodiments of device 200, and obvious variations thereof, is preferred over embodiments of device 100. Manufacturing considerations led to device 200 as the preferred design choice. Basic production principles were applied, including limiting the number of components, and design for ease of molding. These changes led to removal of retention cap 140 from a preferred device design in favor of snap-fit, interlocking features of retention member 260 and element 298 for housing assembly 210 and generator 270. In addition, retaining features across the top of the housing are preferably relieved to allow for injection molding of the housing assembly by removing undercut feature. The rails are preferably kept in off-set locations so that the device cannot be assembled backwards or inverted.

### Device Adaptability to Multiple Commercially Available Generators.

Many commercial CPAP generator devices are available that use a variety of nasal inserts (or patient contacting inserts). These generators typically include variations among both nasal prongs and masks. Embodiments of the housing components for the device are amenable to modification modified to match the specific geometry requirements of which ever generator(s) the manufacturing company would prefer.

The following non-limiting example illustrates the operations of the various sampling systems described herein. The following example generally employs modules and subsystems of the type shown in FIGS. 1 and 3.

### EXAMPLES

Two tests were run to determine the effectiveness of a preferred embodiment (“prototype”). The first test determined if the preferred embodiment's air system performed effectively and the second test measured the force produced on the nose and septum of the infant when the nasal interface was attached.

### Example 1

### Airflow Test Protocol

A test was run to determine the effectiveness of a preferred embodiment (“prototype”). The test measured the force produced on the nose and septum of the infant when the nasal interface was attached.

### Force Test Protocol

The force test was conducted using a specially designed fixture (model test doll). This fixture was based on a doll that was similar to the model used by nurses during training. It was modified to include a pre-calibrated force transducer that was placed in line with the doll's septum. The signal from the transducer was then amplified using an inverting amplifier made using a 741 opamp. Nurses were asked to attach both the Airway's device and prototype to the doll four times each in order to determine the amount of force applied. The testing was conducted in a randomized order to prevent a potential learning curve, or familiarity with the fixture, which could bias the data. For each trial a peak force and steady state force was measured. The steady state force was defined as the average force production for the final five seconds of the trial, after the device is attached, and the fixture is left untouched for five seconds.

### Test Results—Force

TABLE 1
Mean and Standard Deviation of Force Data.

| | PEAK | | STEADY | |
|---|---|---|---|---|
| DEVICE | Mean (lbf)¹ | SD (lbf) | Mean (lbf) | SD (lbf) |
| Prior Art (Airway) | 0.1317072 | 0.185157 | 0.03749 | 0.036811 |
| Prototype | 0.0691347 | 0.055284 | 0.024032 | 0.016171 |

¹lbf, pound force.

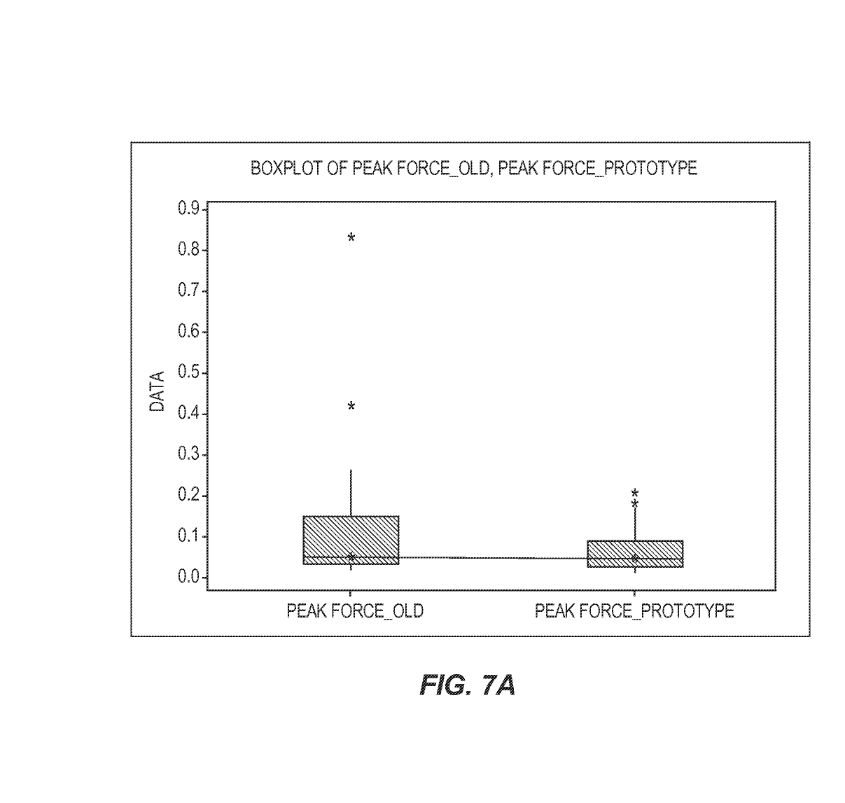

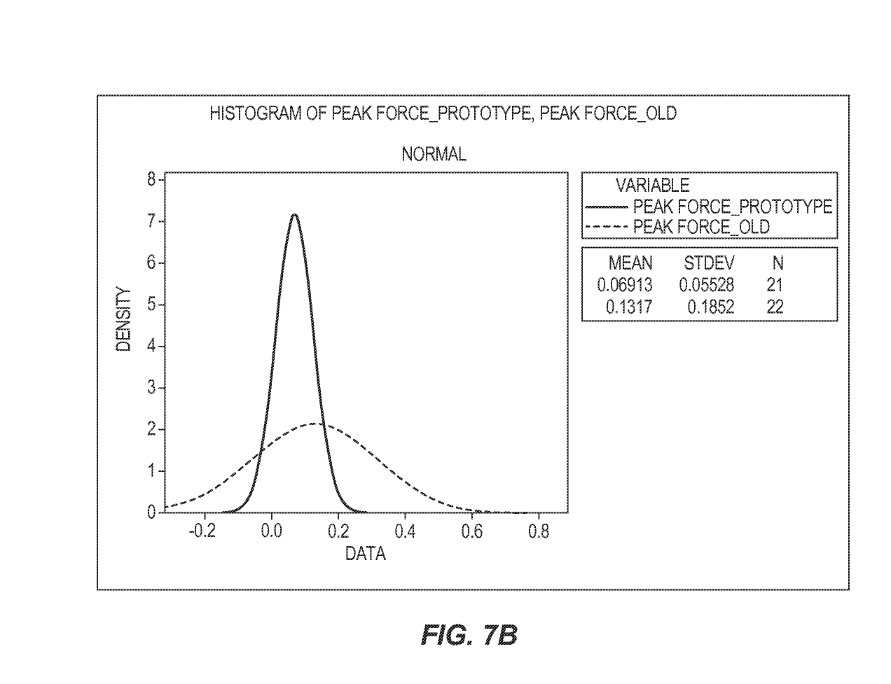

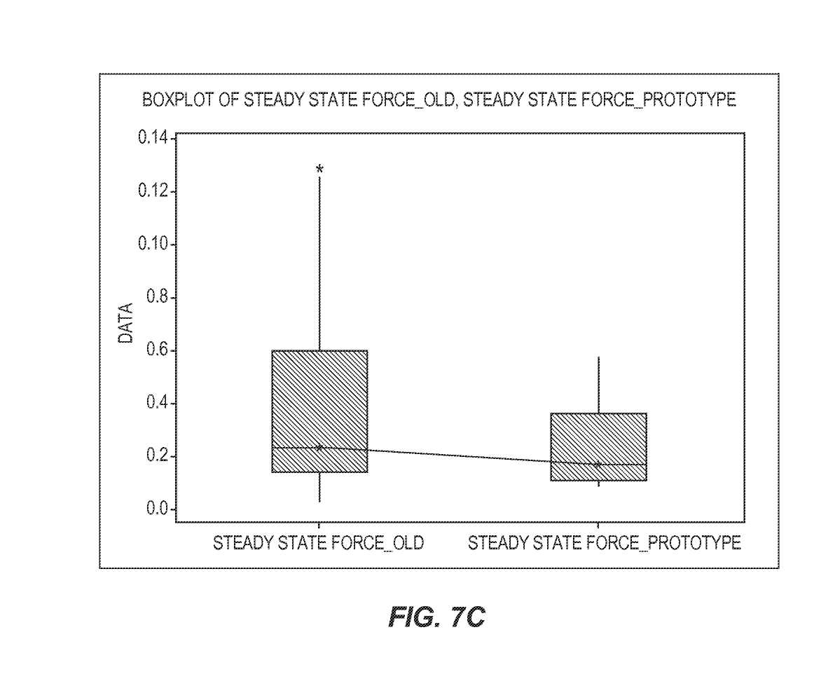

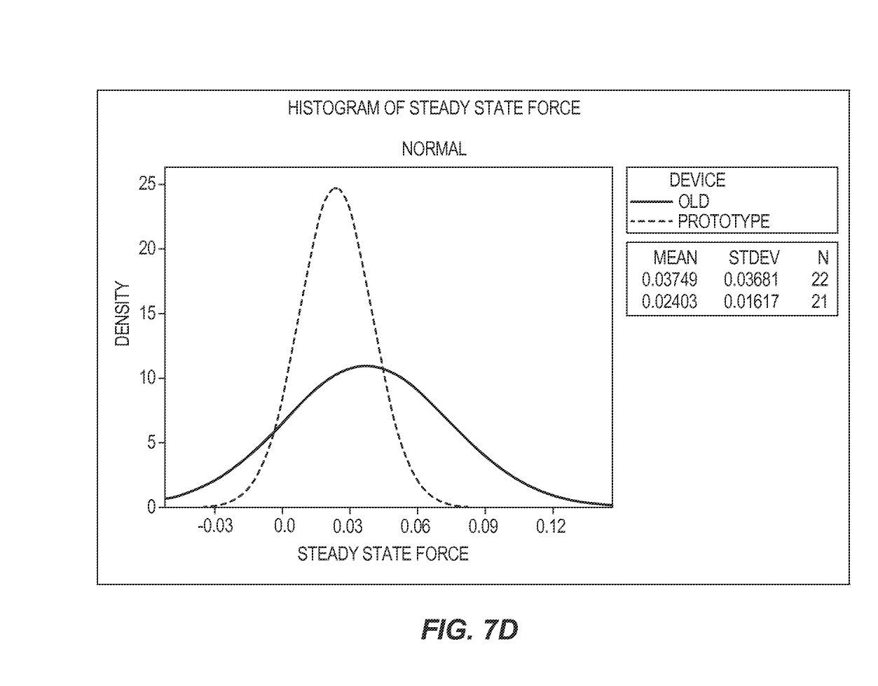

When looking at the data (see FIG. 7), the force applied when using the prototype is lower on average than the force applied when using the Airlife device. It is important to note that by looking at the mean and standard deviation of the data, the standard deviation is much smaller and the spread of the histogram for the prototype is much tighter for the prototype than for the Airlife device. This is very important and promising because it shows that the prototype is more consistent and most importantly avoids extreme outliers (see FIG. 7C) that can potentially damage the infant's septum.

Overall, the data shows that the force applied is lower for the prototype on average. This trend shows proof of concept.

### DEFINITIONS

When introducing elements of aspects of the embodiments, the articles “a,” “an,” “the,” and “said” are intended to mean that there are one or more of the elements. The terms “comprising,” “including,” and “having” are intended to be inclusive and mean that there may be additional elements other than the listed elements. The word “or” means any one member of a particular list and also includes any combination of members of that list, unless otherwise specified.

The modal verb “may” refers to the preferred use or selection of one or more options or choices among several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the model verb “may” has the same meaning and connotation as the auxiliary verb “can.”

The term “about” is used herein to mean approximately, roughly, around, or in the region of When the term “about” is used in conjunction with a numerical range, it modifies that range by extending the boundaries above and below the numerical values set forth. Preferably, the term “about” is used herein to modify a numerical value above and below the stated value by a variance of 20 percent up or down (higher or lower).

The term “guide element” refers to one or more members that couple one or more structures together so that the two structures are slideably engaged, translationally engaged or display the capability of sliding or translating along a path. An example of a guide element is a guide rail or a guide groove.

The term “stop element” refers to one or more members that couple one or more structures together so that the two structures are prevented during one aspect of their coupling path from being slideably engaged, translationally engaged or display the capability of sliding or translating along a path. An example of a stop element is a stop rail or a stop groove. While a stop element might display functional attributes of a guide element, one or more structural features of a stop element prevents the stop element from displaying the full extent of being slideably engaged, translationally engaged or display the capability of sliding or translating along a path.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively or in addition, a component may be implemented by several components.

The above description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

The technical effects and technical problems in the specification are exemplary and are not limiting. It should be noted that the embodiments described in the specification may have other technical effects and can solve other technical problems.

1.-65. (canceled)

66. A force gauged CPAP device, comprising:
a housing assembly;
a spring;
a retention member; and
a generator;
wherein the spring is adapted for coupling to an interior distal wall member of the housing assembly, wherein the generator is disposed slideably within the housing assembly, wherein the retention member maintains the generator within the housing assembly, wherein the spring is adaptively compressed when the generator slideably engages the housing assembly.

67. The force gauged CPAP device of claim 66, wherein the spring is selected from the group consisting of an air spring, a coil spring, a helical spring, a leaf spring, wave spring and a torsional spring.

68. The force gauged CPAP device of claim 66, wherein the retention member comprises an interlocking snap-fit member or a retention cap.

69. The force gauged CPAP device of claim 66, wherein the generator comprises a commercial grade device adapted for treating patients selected from the group consisting of infants and adults.

70. The force gauged CPAP device of claim 66, wherein the spring is coupled to a surface of the housing assembly, to a surface of the generator, or to a surface of both the housing assembly and generator.

71. The force gauged CPAP device of claim 66, further comprising an indicator of the force gauge, wherein the indicator is located for readability either on the top, side or through a window of the force gauged CPAP device

72. The force gauged CPAP device of claim 66, further comprising a plurality of guide elements and stop elements, wherein the plurality of guide elements and stop elements are coupled to one member selected from the group consisting of the housing assembly and the generator or both, wherein the guide elements and stop elements are located either symmetrically or asymmetrically to prevent incorrect assembly of the forced gauged CPAP device.

73. A force gauged CPAP device of claim 66, further comprising an indicator comprising a maximum force indicator that operates translationally relative to the housing and the generator and maintains a position indicative of the maximum force experienced by a patient during use of force gauged CPAP device.

74. A force gauged housing assembly for a CPAP generator, comprising:
a housing assembly;
a spring; and
a retention member;
wherein the spring is adapted for coupling to an interior distal wall member of the housing assembly, wherein the housing assembly is adapted to permit a generator to be slideably disposed within the housing assembly, wherein the retention member is adapted to maintain a generator within the housing assembly, wherein the spring is adapted for compression when a generator slideably engages the housing assembly.

75. The force gauged housing assembly of claim 74, wherein the spring is selected from the group consisting of an air spring, a coil spring, a helical spring, a leaf spring, wave spring and a torsional spring.

76. The force gauged housing assembly of claim 74, wherein the retention member comprises a interlocking snap-fit member or a retention cap.

77. The force gauged housing assembly of claim 74, further comprising at least one indicator of the force gauge.

78. The force gauged housing assembly of claim 77, wherein the at least one indicator of the force gauge is viewable through an indicator window disposed within the housing assembly.

79. The force gauged housing assembly of claim 77, wherein the at least one indicator of force is selected from the group consisting of a real-time indicator and a maximum force indicator.

80. The force gauged housing assembly of claim 74, further comprising a plurality of guide elements and stop elements, wherein the plurality of guide elements and stop elements are coupled to the housing assembly to accommodate one or more different generators.

81. The force gauged housing assembly of claim 80, wherein the plurality of guide elements and stop elements are coupled to housing assembly in a manner to prevent incorrect assembly of force gauged CPAP device when presented with a generator.

82. A maximum force indicator for a CPAP device, comprising:
a plurality of supporting members;
an assembly having a base member and two side wall members, wherein the two side wall members are non-displaceably coupled to the base member, and wherein a plurality of cavities are disposed in the distal portion of the assembly to receive the plurality of supporting members;
a distal wall member having on its inner wall surface at least one push-block member and the plurality of supporting members attached thereto, wherein the distal wall member is displaceably coupled from the assembly; and
a retention member, wherein the retention member maintains the assembly in proximity to a generator when present.

83. The maximum force indicator of claim 82, wherein the supporting members display a coefficient of friction sufficient to maintain the supporting members in an extended position.

84. The maximum force indicator of claim 82, wherein the supporting members include a plurality of measuring indicators.

85. The maximum force indicator of claim 82, wherein the retention member comprises a retention cap or a snap-fit interlocking member.
